(19) United States
(12) Patent Application Publication
(10) Pub. No.: US 2006/0252838 A1
(43) Pub. Date: Nov. 9, 2006

(54) Use of spermine and/or spermidine against skin ageting in dietary, pharmaceuetical or cosmetic compositions

(76) Inventors: Fabio Rinaldi, Milano (IT); Elisabetta Sorbellini, Mediglia (IT); Sergio Baroni, Villa d'Adda (IT); Anna Benedusi, Milano (IT)

Correspondence Address: NIXON PEABODY LLP - PATENT GROUP, CLINTON SQUARE, P.O. BOX 31051, ROCHESTER, NY, 14603-1051, US

(21) Appl. No.: 10/566,411

(22) PCT Filed: Jul. 30, 2004
(86) PCT No.: PCT/EP04/08572

§ 371 Date: Jan. 30, 2006

(30) Foreign Application Priority Data
Jul. 31, 2003 (IT) MI2003A001570

(51) Int. Cl. A61K 31/13; A61K 31/4415; A61K 31/4188; A61K 31/375; A61K 31/28; A61K 31/198; A61K 31/355

(52) U.S. Cl. 514/674; 514/492; 514/562; 514/393; 514/350; 514/474; 514/458

(57) ABSTRACT

The invention concerns the use of spermine and spermidine as active ingredients in the preparation of a composition for dietary, pharmaceutical or cosmetic use in humans for health and beauty of the skin and skin appendages, and related compositions for pharmaceutical or dietary or cosmetic use for administration to humans.

### DESCRIPTION

The present invention concerns a new use of the polyamines called spermine (N,N′-bis(3-aminopropyl)tetramethylendiamine) and spermidine (N-(3-aminopropyl)tetramethylendiamine).

It is known in literature that compounds belonging to the class of aliphatic polyamines perform a decisive role in control of the biological mechanisms of growth, division, differentiation of cells and proliferation of animal tissues.

The polyamines in question comprise essentially the compounds putrescine, spermine and spermidine. The latter two owe their name to the fact that they were discovered for the first time in human sperm. In reality spermidine is present in practically all the body fluids (blood, saliva, tears, milk). Subsequently spermidine was found also in many foods both of animal origin (meat, fish, eggs, milk, cheese) and vegetable origin (fruit and vegetables). Its concentration is particularly high in human milk (on average approximately 600 micrograms in milk over a 24-hour period) where it performs a very important function for babies. In babies, in fact, the mucous membranes of the digestive tract are not perfectly formed and the spermidine contained in the milk promotes growth of the epithelium of the gastric and intestinal mucous membranes.

Spermine derives biosynthetically from spermidine, via the action of specific amino-propylic radical donor enzymes, which transform the putrescine, common precursor, firstly into N-monoaminopropyl derivative (spermidine) and then into N,N′-diaminopropyl symmetric derivative (spermine). Spermidine is therefore the biosynthetic precursor of spermine.

Spermidine and spermine therefore represent important cell growth and proliferation factors.

According to the present invention it has now surprisingly been found that a preparation containing spermine or spermidine, whether administered orally or applied to the skin, stimulates the cells of the skin and skin appendages such as hairs, hair and nails, with consequent promotion of growth and regeneration of the cells. The consequence is an effect that improves both the appearance and functional characteristics of the skin and skin appendages and combats ageing.

The subject of the present invention is therefore use of the polyamines spermine and spermidine, as is or in salified form, as the active ingredient in preparation of compositions for dietary, pharmaceutical or cosmetic use in humans, aimed at maintaining health and beauty of the skin and skin appendages and combating ageing.

The subject of the present invention is also a composition for pharmaceutical, dietary or cosmetic use for use in humans to maintain health and beauty of the skin and skin appendages and combat ageing, characterised in that it comprises as active ingredient spermine, spermidine or their salts.

Said composition can comprise as active ingredient spermine or spermidine or both, in free or salified form.

For a better understanding of the characteristics and advantages of the invention, the details of an experimental study giving rise to said invention are now described.

The Clinical Study

The study determined some of the fundamental indexes of health and functionality of the skin and skin appendages. In order to verify the effect of the substances being studied, the following parameters, considered to be of great importance, were identified and assessed:

hydration

elasticity

cell renewal

Assessment of Hydration and Elasticity

The effectiveness of the product was assessed in vivo by testing in use, carried out on 20 adult consenting volunteers (aged between 18 and 55).

On the forearms of each volunteer 3 areas were selected:
one for application of the product being studied containing spermidine;
one for application of the product being studied without spermidine;
one as a control area.

A composition for topical use according to the invention (composition containing spermidine) and a product without spermidine (placebo) are given to the subjects who will apply them, according to the procedures indicated above, twice a day for 1 month.

At the beginning and at the end of the test the following instrumental assessments of effectiveness are performed:
skin hydration by means of comeometer
skin elasticity by means of cutometer

For each area (product, placebo, control) the values recorded at the beginning of the test were compared, via appropriate statistical processing, with the data obtained at the end of the test. The variations obtained in the area treated with the product were further compared with those recorded at the place of application of the placebo.

The results showed an increase in skin hydration with a statistically significant difference between the mean values observed after treatment and the corresponding values observed after the placebo. The degree of hydration, determined by electric capacitance measured with the corneometer, increased by over 10% with a high statistical significance (p<0.001).

The values recorded with the cutometer highlighted also in this parameter statistically significant differences (+20%; p<0.001) between the elasticity values before and after the treatment, also taking account of the effect due to the placebo preparation.

Assessment of Cell Renewal

On the forearms of each volunteer 3 areas were selected, on each of which a 5% suspension of dansyl chloride in vaseline was applied (with occlusive bandaging for 20±4 hours). The following day the patches were removed and the 3 skin areas were examined under a quartz UV lamp to assess the degree of fluorescence induced by the dansyl chloride. Using a numerical reference scale, a score was assigned to the intensity of each spot.

The subjects were then given the composition of the invention and the placebo, with the recommendation to apply them as follows:
in the first area the product containing spermidine;
in the second area the product without spermidine;
in the third area no product as it is the control area.

The volunteers applied the samples twice a day and were recalled regularly to the laboratory until complete disappearance of the fluorescent spots. At the beginning and end of the test, corresponding to the 2 areas selected, the quantity of superficial comeocytes was measured by means D-Squame (transparent adhesive discs).

The effectiveness of cell renewal was expressed as the number of days required to induce disappearance of the fluorescence in the areas treated (with the product or with the placebo) with respect to the control area. The statistical analysis highlighted shortening of the cell renewal period in the order of 20% (p<0.01).

### EXAMPLES

Some non-restrictive examples of the composition according to the invention are now described.

### Example 1

Dietary Composition for Oral Use for Health and Beauty of Skin and Nails Tablets

Each tablet contains:

| | | |
|---|---|---|
| Methyl sulfonyl methane | 200 | mg |
| Spermidine trihydrochloride | 0.25 | mg |
| Vitamin C | 61.86 | mg |
| Vitamin E (dl-alfa tocopherol) | 32.89 | mg |
| Vitamin B6 (Pyridoxine) | 3.65 | mg |
| Calcium d-Panthotenate | 4 | mg |
| d-Biotin | 0.23 | mg |
| Zinc aminoacid chelate | 37.5 | mg |
| Copper aminoacid chelate | 12 | mg |
| Manganese aminoacid chelate | 22.5 | mg |
| Selenium yeast 2000 μg/g | 13.75 | mg |
| Microcrystalline cellulose | 120 | mg |
| Calcium phosphate dibasic dihydrate | 98.89 | mg |
| Hydroxypropyl methylcellulose | 52.5 | mg |
| Magnesium stearate | 8 | mg |
| Silicon dioxide | 3.5 | mg |

### Example 2

Dietary Composition for Oral Use for Health and Beauty of Skin Exposed to Radiation Tablets

Each tablet contains:

| | | |
|---|---|---|
| Spermidine trihydrochloride | 0.25 | mg |
| Calcium panthotenate | 4 | mg |
| Ubidecarenone | 10 | mg |
| Vitamin C | 62 | mg |
| Vitamin E (dl-alfa tocopherol) | 33 | mg |
| Beta-Carotene | 36 | mg |
| Vitamin B6 (Pyridoxine) | 3.65 | mg |
| d-Biotin | 0.225 | mg |
| Zinc aminoacid chelate | 37.5 | mg |
| Copper aminoacid chelate | 12 | mg |
| Manganese aminoacid chelate | 17.5 | mg |
| Calcium phosphate dibasic dihydrate | 120 | mg |
| Microcrystalline cellulose | 259.38 | mg |
| Hydroxypropyl methylcellulose | 56 | mg |
| Magnesium stearate | 7 | mg |
| Silicon dioxide | 1.75 | mg |

### Example 3

Cosmetic Composition for Topical Skin Treatment Emulsion

100 ml of emulsion contain:

| | | |
|---|---|---|
| Spermidine trihydrochloride | 0.02 | g |
| Emulgade SE (Glyceryl Stearate, Ceteareth-20, | 4.5 | g |
| Ceteareth-12, Cetearyl alcohol, Cetyl palmitate) | | |
| Ceteareth | 201 | g |
| Coco-caprylate/caprate | 5 | g |
| Dicaprylyl ether | 5 | g |
| Water | q.s. to 100 | ml |

### Example 4

Cosmetic Composition for Topical Skin Treatment With Sun Filter

Lotion Applicable Also in Spray

100 ml of lotion contain:

| Spermidine trihydrochloride | 0.01 | g |
|---|---|---|
| Emulgade SE (Glyceryl Stearate, Ceteareth-20, | 3.9 | g |
| Ceteareth-12, Cetearyl alcohol, Cetyl palmitate) | | |
| Ceteareth | 203.1 | g |
| Coco-caprylate/caprate | 7 | g |
| Octyl methoxycinnamate | 4 | g |
| Isoamyl methoxycinnamate | 6 | g |
| Benzophenone-3 | 2 | g |
| Tocopherol | 0.5 | g |
| Glycerol | 5 | g |
| Preservative, fragrance | q.s. | |
| Water | 64.5 | g |

1. Use of spermine and/or spermidine in free or salified form as active principle in the preparation of a composition for dietary, pharmaceutical or cosmetic use in humans for health and beauty of the skin and skin appendages, to combat ageing thereof.

2. Use of spermine and/or spermidine in free or salified form as active principle in the preparation of a composition for dietary, pharmaceutical or cosmetic use in humans so as to improve at least one of the following properties of the human skin: hydration, elasticity, cell renewal.

3. Composition for pharmaceutical or dietary or cosmetic use for administration in humans for health and beauty of the skin and skin appendages characterised in that it comprises as active principle spermine or spermidine or both, in free or salified form.

4. Composition for pharmaceutical or dietary or cosmetic use for administration in humans so as to improve at least one of the following properties of the human skin: hydration, elasticity, cell renewal, characterised in that it comprises as active principle spermine or spermidine or both, in free or salified form.

5. Composition according to claim 3, characterised in that it also comprises methyl sulfonyl methane or methionine, vitamin C, vitamin E, Vitamin B6, calcium d-panthotenate, d-biotin, zinc (as aminoacid chelate), copper (as aminoacid chelate), manganese (as arninoacid chelate) and a source of organic selenium.

6. Composition according to claim 3, characterised in that it comprises:
Methyl sulfonyl methane 200 mg
Spermidine trihydrochloride 0.25-0.5 mg
Vitamin C 60-90 mg
Vitamin E (d1-alfa tocopherol) 33 mg
Vitamin B6 (Pyridoxine) 3.7 mg
Calcium d-Panthotenate 4 mg
d-Biotin 0.23 mg
Zinc (as aminoacid chelate) 7.5 mg
Copper (as aminoacid chelate) 1.25 mg
Manganese (as aminoacid chelate) 2.25 mg
Selenium (as Se yeast) 0.03 mg

7. Composition according to claim 3, characterised in that it is suitable for oral administration.

8. Composition according to claim 3, characterised in that it is suitable for topical administration, such as a lotion or cream.

9. Composition according to claim 4, characterised in that it also comprises methyl sulfonyl methane or methionine, vitamin C, vitamin E, Vitamin B6, calcium d-panthotenate, d-biotin, zinc (as aminoacid chelate), copper (as aminoacid chelate), manganese (as aminoacid chelate) and a source of organic selenium.

10. Composition according to claim 4, characterised in that it comprises:
Methyl sulfonyl methane 200 mg
Spermidine trihydrochloride 0.25-0.5 mg
Vitamin C 60-90 mg
Vitamin E (d1-alfa tocopherol) 33 mg
Vitamin B6 (Pyridoxine) 3.7 mg
Calcium d-Panthotenate 4 mg
d-Biotin 0.23 mg
Zinc (as aminoacid chelate) 7.5 mg
Copper (as aminoacid chelate) 1.25 mg
Manganese (as aminoacid chelate) 2.25 mg
Selenium (as Se yeast) 0.03 mg

11. Composition according to claim 4, characterised in that it is suitable for oral administration.

12. Composition according to claim 4, characterised in that it is suitable for topical administration, such as a lotion or cream.
